(19) United States
(12) Patent Application Publication
(10) Pub. No.: US 2009/0093684 A1
(43) Pub. Date: Apr. 9, 2009

(54) SURGICAL RETRACTOR DEVICE AND METHOD OF USE

(76) Inventor: Scott Schorer, Duxbury, MA (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/868,843

(22) Filed: Oct. 8, 2007

(51) Int. Cl. A61B 1/32; A61B 17/04; A61B 19/00

(52) U.S. Cl. 600/210; 600/201; 600/219; 600/210; 606/301; 128/898

(57) ABSTRACT

A surgical retractor is disclosed. The retractor has a body with first and second portions cooperating to displace circularly relative to one another about an axis through a center of the body. A first retractor blade attaches to the first portion of the retractor body, and a second retractor blade attaches to the second portion of the retractor body. The first and second retractor blades define an angle therebetween and the angle is adjusted by the circular displacement of the first and second body portions

### CROSS REFERENCE TO RELATED APPLICATIONS

This application claims priority to U.S. Provisional Patent Application No. 60/821,541, filed Oct. 6, 2006, and entitled SURGICAL RETRACTOR DEVICE AND METHOD OF USE, which is hereby incorporated by reference.

### BACKGROUND

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae (twenty-four in all) separated by intervertebral discs. These discs hold the vertebrae together in a flexible manner so as to allow relative movement between the vertebrae from front to back and from side to side. This movement allows the body to bend forward and backward, to twist from side to side, and to rotate about a vertical axis. Throughout this movement, when the spine is operating properly, the nerves are maintained clear of the hard structure of the spine.

Over time or because of accidents, the intervertebral discs tend to lose height or become cracked, dehydrated, or herniated. The result is that the height of one or more discs may be reduced, which may lead to compression of the nerve bundles. Such compression may cause pain and, in some cases, damage to the nerves.

Currently, there are many systems and methods at the disposal of a physician for reducing or eliminating the pain by minimizing the stress on the nerve bundles. In some instances, the existing disk is removed and an artificial disk is substituted therefore. In other instances, two or more vertebrae are fused together to prevent relative movement between the fused discs.

Often there is required a system and method for maintaining or recreating proper space for the nerve bundles that emerge from the spine at a certain location. In some cases, a cage or bone graft is placed in the disc space to preserve or restore height and to aid in fusion of the vertebral level. As an aid in stabilizing the vertebrae, one or more rods or braces are placed between the fused vertebrae with the purpose of supporting the vertebrae, usually along the posterior of the spine, while fusion takes place. These rods are often held in place by anchors that are placed into the pedicle of the vertebrae.

Minimally invasive surgical procedures have been developed to fuse the vertebrae. Such procedures can reduce pain, post-operative recovery time, and the destruction of healthy tissue. Generally, a pathological site is accessed through portals rather than through a significant incision, which aids in preserving the integrity of the intervening tissues. Minimally invasive surgical procedures are particularly desirable for spinal and neurosurgical applications because of the need for access to locations deep within the body and the possible range of damage to vital intervening tissues. In such procedures, however, it may be necessary to hold the edges of an incision apart to provide a clear operating field within which the surgeon can operate.

What is needed, therefore, is a tool or retractor adapted to work with minimally invasive procedures that allows the surgeon to have a clear path to the operating field, and a method for using such a tool or retractor.

### BRIEF DESCRIPTION OF THE DRAWINGS

FIG. 1a is a perspective view of one embodiment of a retractor positioned with respect to a layer of tissue.

FIG. 1b is a perspective view illustrating one embodiment of a retractor.

FIG. 1c is a perspective view of the retractor of FIG. 1b with a portion of the retractor removed.

FIG. 2 is a perspective view of a portion of the retractor of FIG. 1b.

FIG. 3 is a side view of the retractor of FIG. 1a taken along lines 3-3.

FIG. 4 is a side view of the retractor of FIG. 1a taken along lines 4-4.

FIG. 5 is a side view of the retractor of FIG. 4 with the addition of a blade.

FIG. 6a is a side view of the retractor of FIG. 1b taken along lines 4-4 with the addition of a blade.

FIG. 6b is a side view of the blade of FIG. 6a.

FIG. 7a is a side view of the retractor of FIG. 1b taken along lines 4-4 with the addition of a blade.

FIGS. 7b and 7c are side views of two different embodiments of a blade that may be used with the retractor of FIG. 7a.

FIG. 8a is a side view of another embodiment of a retractor with a blade.

FIG. 8b is a side view of the blade of FIG. 8a.

FIGS. 9a-9d illustrate various embodiments of blades that may be used with the retractor of FIG. 1b.

FIG. 10a is a top view of the retractor of FIG. 1b in a first or substantially collapsed position.

FIG. 10b is a top view of a retractor similar to that of FIG. 10a in a second or partially open position.

FIG. 11 is a perspective view of an embodiment of the retractor of FIG. 1b in a first or substantially collapsed position while being used with a spinal surgery system.

FIG. 12a is a perspective view of the retractor of FIG. 11 illustrating an exemplary positioning of the retractor with respect to a layer of tissue.

FIG. 12b is a top view of the retractor of FIG. 12a illustrating an exemplary positioning of the retractor with respect to the layer of tissue prior to the insertion of a rod between two extensions.

FIG. 12c is a top view of the retractor of FIG. 12a illustrating an exemplary positioning of the retractor with respect to the layer of tissue after the insertion of a rod between two extensions.

FIGS. 13-15 are side views illustrating possible orientations of the retractor of FIG. 12.

FIG. 16 is a perspective view of an embodiment of the retractor of FIG. 11 in a second or substantially open position.

FIG. 17 is a side view of one embodiment of an instrument that may be used to move a retractor from the substantially collapsed position of FIG. 11 to the substantially open position of FIG. 16.

FIG. 18 is a cross-sectional view of another embodiment of a retractor.

### DETAILED DESCRIPTION OF THE PREFERRED EMBODIMENTS

The present disclosure is directed to systems and methods for retracting tissues in surgical procedures. It is understood that the following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

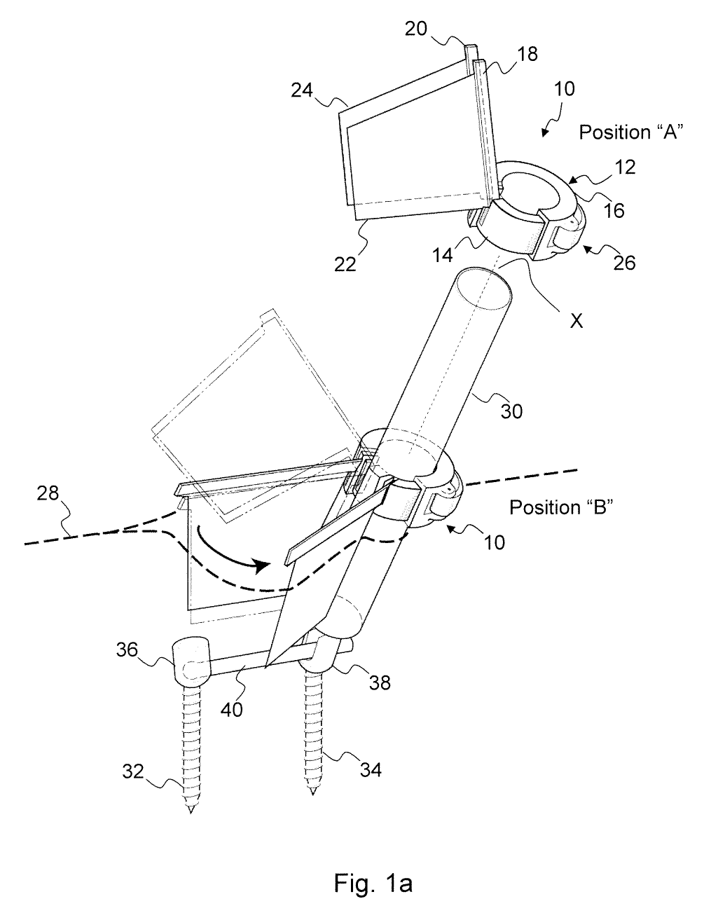

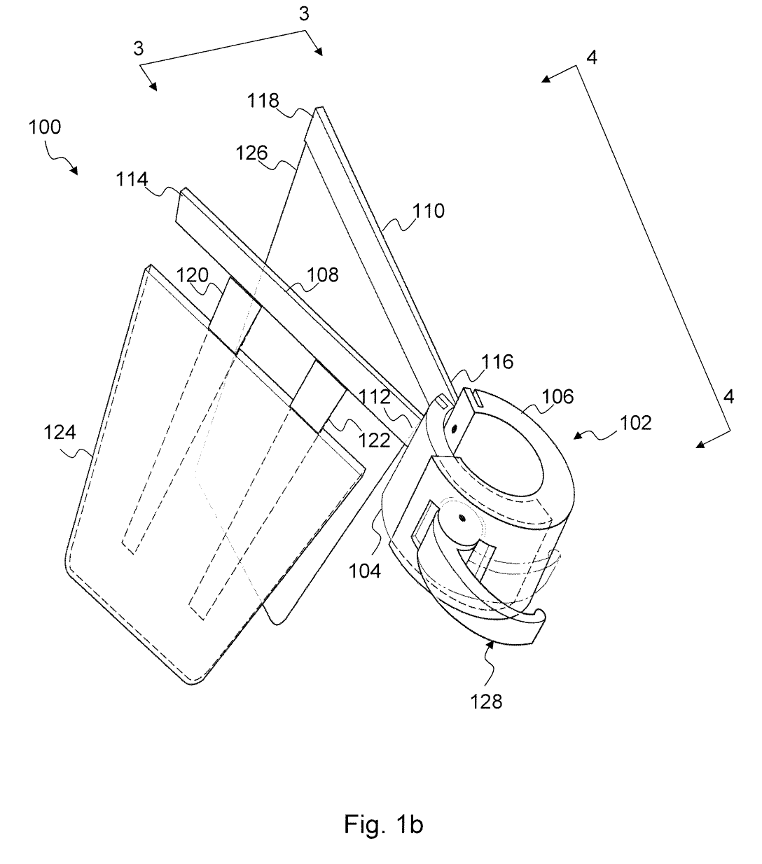

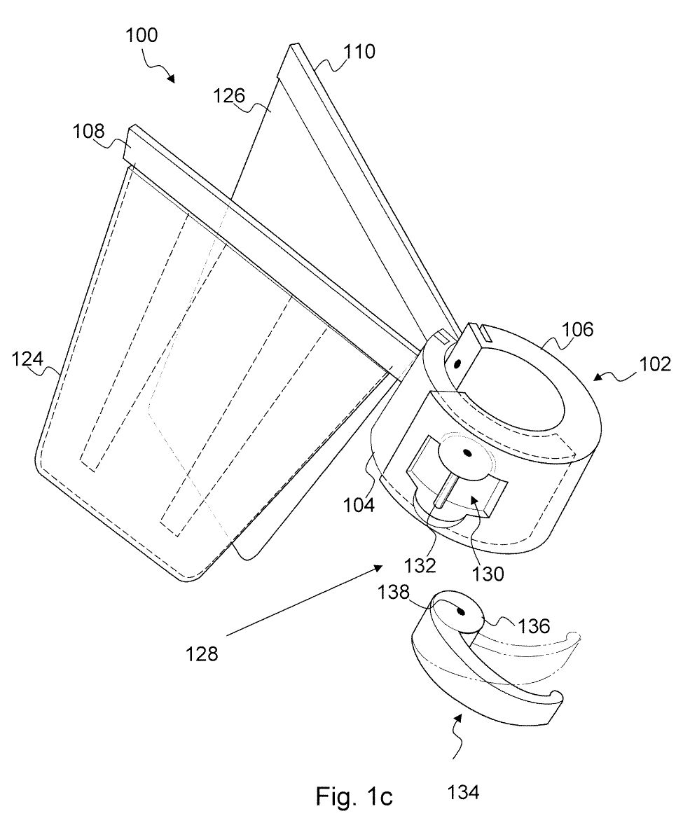

Referring to FIG. 1a, in one embodiment, a retractor 10 is illustrated in conjunction with an extension 30, such as may be used in a spinal surgery procedure. As illustrated, the retractor 10 may include a substantially circular body 12 formed by semi-circular body portions 14 and 16 that may move relative to one another, as will be discussed later in greater detail. The body portions 14 and 16 may be coupled to arms 18 and 20, respectively, that extend outwardly from the circular body 12. Each arm 18 and 20 may be coupled to a blade 22 and 24, respectively, which may be used to provide an opening at a surgical site. A locking mechanism 26 may be used to lock the relative positions of the body portions 14 and 16.

In the present example, bone anchors 32 and 34 (e.g., pedicle screws) may be fastened to a vertebral body (not shown). The bone anchors 32 and 34 may have polyaxial heads 36 and 38, respectively, which may be configured to receive a rod 40. Examples of the extension 30 and its interaction with the bone anchors 32, 34, polyaxial heads 36 and 38, and rod 40 are illustrated in greater detail in U.S. patent application Ser. No. 10/690,211, filed on Oct. 21, 2003, U.S. patent application Ser. No. 10/990,272, filed on Nov. 16, 2004, and U.S. patent application Ser. No. 10/989,715, filed on Nov. 16, 2004, all of which are hereby incorporated by reference in their entirety.

In operation, the retractor 10 may be placed in a position “A” above the extension 30. As illustrated, the arms 18 and 20 may be rotated partially or totally upward. The circular body 12 of the retractor 10 may then be placed over the extension 30 and may slide down the extension to a position “B”. At this point, the arms 18 and 20 may be lowered. It is noted that the locking mechanism 26 may be manipulated to allow movement of the body portions 14 and 16 with respect to one another and to prevent such relative movement.

As illustrated, after the arms 18 and 20 are lowered while the retractor 10 is in position “B”, a portion of the blades 22 and 24 may be positioned beneath a tissue layer 28. For example, the tissue layer 28 may be a layer of skin, with the extension 30 positioned within an incision in the tissue layer. As illustrated, an upper portion of the extension 30 may be above the tissue layer 28 and a lower portion below the tissue layer. Accordingly, the retractor 10 may be used to enlarge an opening below the tissue layer 28.

It can also be seen in FIG. 1a that the angle of the arms 18 and 20, and therefore the blades 22 and 24, may be varied with respect to the body 12. As will be described in greater detail below, this may allow the angle of the arms 18 and 20, and blades 22 and 24, to vary with respect to a longitudinal axis X passing through the body 12. Such a longitudinal axis is substantially coaxial with the extension 30 inserted through the body 12.

The angle between the arms 18 and 20, and the blades 22 and 24 also varies with the relative adjustment between the two body portions 14 and 16 of the body 12. The angle between the arms 18 and 20 may be adjustable from substantially no separation, as shown in position “A”, to an acute angle as shown in position “B”. Some embodiments may allow the arms 18 and 20, and blades 22 and 24 to adjust to a position with an obtuse angle between the arms 18 and 20.

Referring to FIG. 1b, a perspective view of one embodiment of a retractor 100 is provided. As illustrated, the retractor 100 may include a substantially circular body 102 formed by semi-circular body portions 104 and 106. In certain embodiments, the body portions 104 and 106 may move relative to one another in a sliding manner. For example, the body portion 106 may include a cavity large enough to receive at least part of the body portion 104 such the body portion 104 slides within and interfits with the body portion 106. Accordingly, the body portions 104 and 106 may move with respect to one another, and such movement may be restrained to a substantially circular path. In the present embodiment, the body portion 104 may not be removable from the body portion 106 due to a length of the body portion 104 captured within the cavity of the body portion 106 and due to features captured within to prevent disassembly.

In other embodiments, the general shape of the body 102 may be generally cylindrical with each of the body portions 104 and 106 for a portion of the generally cylindrical shape. In another embodiment, the body 102 may be generally toroidal, donut shaped, or shaped as an annulus. In each of these additional embodiments, the body portion 106 may accommodate the body portion 104 in a sliding interfit relationship, as described. The path of the body portion 104 within the body portion 106 may remain substantially circular. In another embodiment, the body portion 106 may substantially cover the body portion 104. A window (not shown) on the body portion 106 may be provided allowing circular translation and longitudinal rotation of the arm 102.

The body portions 104 and 106 may be coupled to arms 108 and 110, respectively, that extend outwardly from the circular body 102. More specifically, a inward end 112 and 116 of each arm 108 and 110, respectively, may be coupled to their respective body portion 104 and 106. As will be described later, a distal end 114 and 118 of each arm 108 and 110, respectively, may be moved as the proximal ends 112 and 116 are rotated.

As illustrated, in certain embodiments, the arm 108 may include one or more projections 120 and 122 that may extend substantially perpendicularly from a longitudinal axis of the arm. Although not shown in FIG. 1b, it is understood that the arm 110 may include similar projections.

In certain embodiments, the blade 124 may be coupled to the projections 120 and 122 of the arm 108, and a blade 126 may be coupled to the similar projections (not shown) of the arm 110. In the present example, one or both of the blades 124 and 126 may be removable, and may be reusable or single use (i.e., molded plastic). For example, the blade 124 may include one or more slots for receiving the projections 120 and 122.

It is understood that the term “blade” as used herein does not necessarily imply a cutting edge, and the various edges of a blade may or may not be designed for cutting. For example, the blade 124 may be tapered, with a thicker flat-edged portion near the arm 108 and a narrower round-edged portion at the opposite edge. As will be illustrated later, such a taper may aid in inserting the blade into a surgical site. However, such tapering is not required, and the blade may have any desired shape. Furthermore, the thickness of the blade 124 may vary depending upon the needs of the user. The blade 124 may also have various concavities, convexities, lips, contours and other features promoting efficient retraction within the confines of the prevailing anatomy at a surgical site.

In the present example, as will be described later in greater detail, the circular body 102 may include a mechanism 128 for locking a position of the body portion 104 relative to that of the body portion 106. In certain embodiments, locking the body portion 104 relative to the body portion 106 also locks the retractor 100 to an extension, such as the extension 30 of FIG. 1a. In other embodiments, the body portion 106 may have vertical grooves or channels (not shown) which slidingly engage with protrusions on the extension to keep the body portion 106 stationary with respect to the extension. In yet other embodiments, protrusions on the inside surface of the body portion 106 may slidingly engage channels or grooves on the exterior surface of the extension.

Referring to FIG. 1c, the circular body 102 is illustrated with a portion of the mechanism 128 removed. In the present example, the mechanism 128 may include an opening 130 in the body portion 104. In certain embodiments, the opening 130 may extend through the body portion 106 to reveal part of the body portion 104. A pin 132 may span the opening 130 to secure a lever 134 to the body portion 106.

In certain embodiments, the lever 134 may include an enlarged portion 136 that may be shaped to apply pressure to the underlying body portion 106 when in a first position and to release the pressure when in a second position. Such an arrangement may be considered a camming fit with the enlarged portion 136 operating as the cam lobe. The lever 134 may lock the relative positions of the body portions 104 and 106 in a first position (e.g., when down or when proximate to the body portion 104) and may allow movement in a second position (e.g., when away from the body portion or when up). In another embodiment, the lever may lock the relative positions of the body portions 104 and 106 when up, but allow movement when down (e.g., proximate the body portion). The pin 132 may extend through a bore 138 in the lever 134 to secure rotation of the lever 134.

In other embodiments, different mechanisms for locking the body portions 104 and 106 could be utilized and the camming arrangement shown in FIG. 1c is meant to be illustrative only. In some embodiments, a series of holes defined in the body portions 104 may allow pins to be used to secure the body portions on the proper relative locations once the desired retraction has been achieved. In other embodiments, a racketing mechanism could be utilized where the arms 108 and 110 are retained in the largest angle that they are moved into until released. In further embodiments, the retraction strength needed of the retractor 100 may be slight enough to allow a friction or interference fit between the body portions 104 and 106.

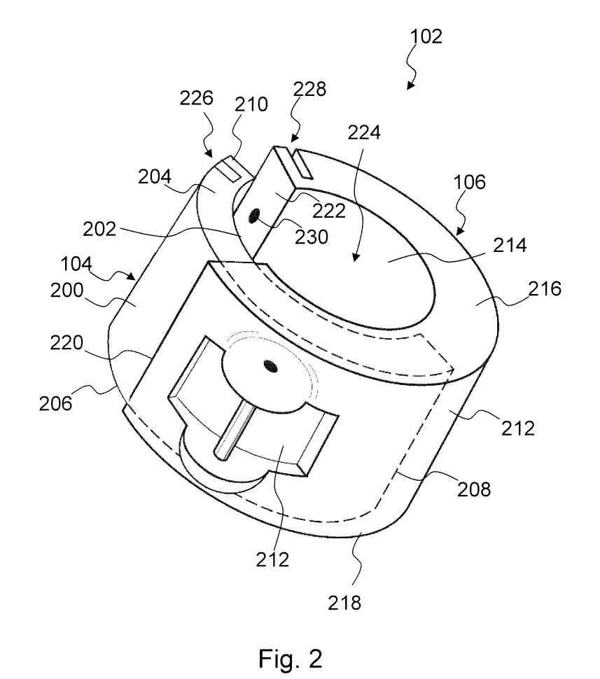

Referring to FIG. 2, in one embodiment, the body 102 is illustrated without the arms 108 and 110 (FIG. 1b). In the present example, the body portion 104 includes an exterior surface 200 and an interior surface 202. The exterior and interior surfaces 200 and 202 may be separated by an upper surface 204 and a lower surface 206. The upper and lower surfaces 204 and 206 may have the same or different widths (e.g., the exterior and interior surfaces 200 and 202 may taper upward toward the upper surface 204 or downward toward the lower surface 206). The body portion 104 may also have an interior end 208 that is inserted into the cavity of the body portion 106 and an exterior end 210 that is opposite the end 208.

The body portion 106 includes an exterior surface 212 and an interior surface 214. The exterior and interior surfaces 212 and 214 may be separated by an upper surface 216 and a lower surface 218. The upper and lower surfaces 216 and 218 may have the same or different widths (e.g., the exterior and interior surfaces 212 and 214 may taper upward toward the upper surface 216 or downward toward the lower surface 218). The body portion 106 may also have a cavity end 220 that opens into the cavity of the body portion 106 and an exterior end 222 that is opposite the end 220.

The interior surface 214 of the body portion 106 may be semi-circular and, in conjunction with the interior surface 202 of the body portion 104, may provide a substantially circular or cylindrical opening 224. Although the opening 224 in the present embodiment may be relatively fixed in size due to the inner surfaces 202 and 222, it is understood that flexibility in the size of the opening may be provided in other embodiments. Furthermore, the exact shape of the opening 224 may vary in other embodiments as the general shape of the body 102 differs in other embodiments, (e.g., a toroidal body)

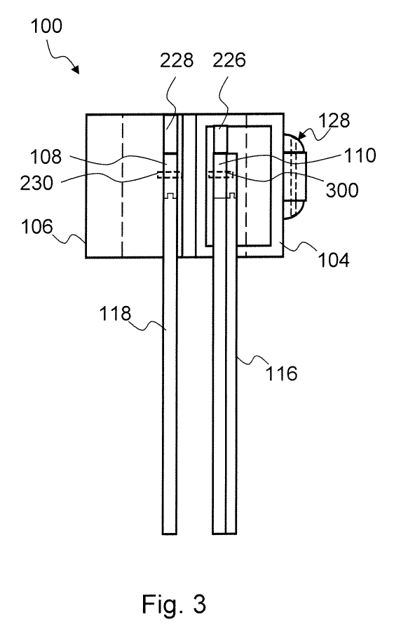

In some embodiments, the exterior end 210 may include a channel 226 and the exterior end 222 may include a channel 228. The channels 226 and 228 may be sized to receive the arms 108 and 110, respectively. A pin (see FIG. 3) may span the channel 226 and a pin 230 may span the channel 228. The pins may couple the arms 108 and 110 to the body portions 104 and 106, respectively. The channels 226 and 228 may enable the arms 108 and 110 to rotate upward around an axis provided by each pin. The angular positions of the arms with respect to a longitudinal axis proceeding through the body 102 may be maintained by friction between the arms 108 and 110 and the channels 226 and 228. In other embodiments, the arms 108 and 110 may be held in place by other means or may be free to rotate relatively freely.

The arc through which each arm 108 and 110 may be rotated may be sufficient to move the arms from a first position to a second position. For example, the first position may be where a longitudinal axis of one or both arms 108 and 110 is substantially aligned with the longitudinal axis extending through the center of the opening 224. This longitudinal axis extending through the center of the opening 224 may also be the longitudinal axis of the extension 30 (FIG. 1a) when inserted through the body 102. The second position may be where the longitudinal axis of one or both arms 108 and 110 is separated from the first position by an acute angle, a right angle, or an obtuse angle. It is understood that the arms 108 and 110 may be rotated independently or together.

Referring to FIG. 3, one embodiment of a side view of the retractor 100 of FIG. 1b is illustrated, although FIG. 3 differs from FIG. 1b in that the arm 108 has been moved into a substantially parallel alignment with the arm 110. In the present example, each body portion 104 and 106 may be coupled to its respective arm 108 and 110, respectively, by means of pins 300 and 230, respectively. As described previously, the body portions 104 and 106 may include the channels 226 and 228, respectively, and the pins 300 and 230 may span their channel.

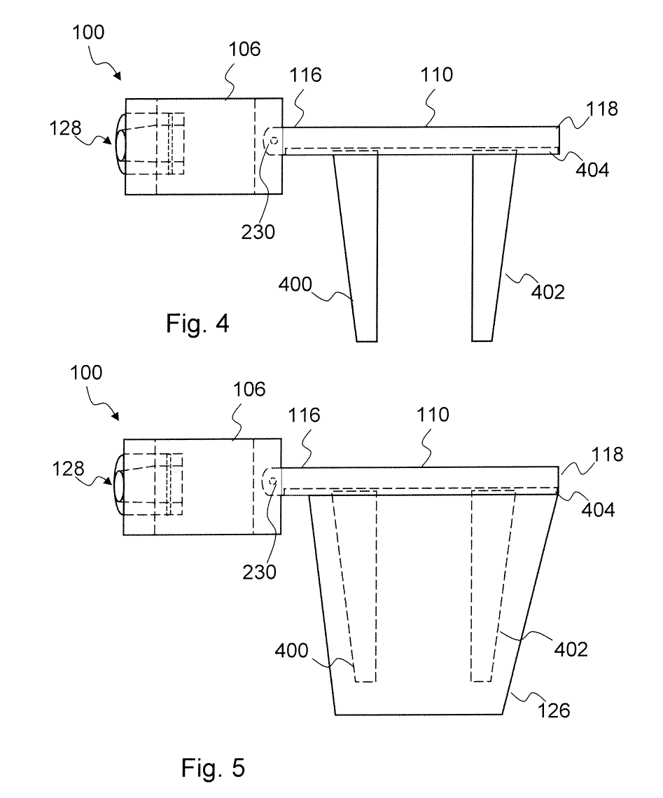

Referring to FIG. 4, an embodiment of a side view of the retractor of FIG. 1b is illustrated. As described previously, the arm 110 may include two projections 400 and 402. The projections 400 and 402 may be fixed, removable, and/or adjustable, and the arm 110 may include a slot or channel 404 configured to receive the projections. For example, the projections 400 and 402 may snap into the channel 404, or the channel 404 may have an opening at one end into which the projections may be inserted (e.g., in a tongue and groove fitting). In the present embodiment, the projections 400 and 402 may be slidably attached to the arm 110 via the channel 404. It is understood that the projections 400 and 402 may be fastened to the arm 110 in many different ways, and those described are for purposes of illustration only.

Referring to FIG. 5, the retractor 100 of FIG. 1b is illustrated with the addition of the blade 126 (FIG. 1b).

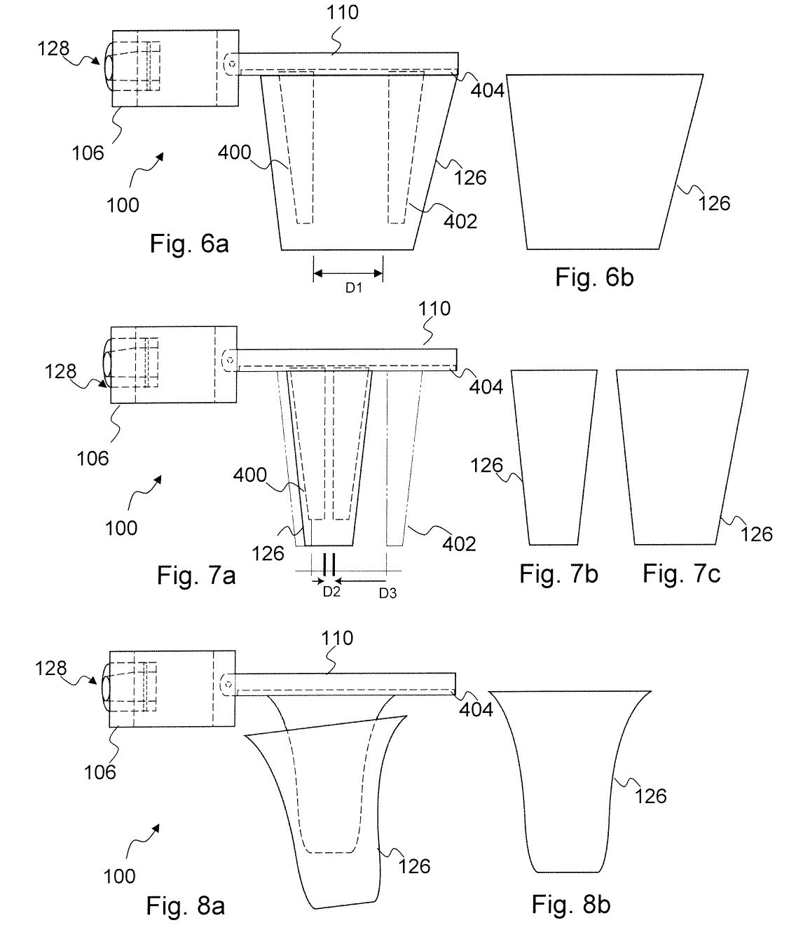

Referring to FIGS. 6a and 6b, in one embodiment, the retractor of FIG. 1b is illustrated with a distance D1 separating the projections 400 and 402. The distance D1 may be selected to provide support for the blade 126 and may coincide with the spacing of slots in the blade. In other embodiments, the slots may be relatively large compared to the width of the projections 400 and 402. In still other embodiments, the blade 126 may have a single slot into which both projections 400 and 402 are inserted.

Referring to FIGS. 7a-7c, in another embodiment, the retractor of FIG. 1b is illustrated with the projections 400 and 402 separated by varying distances smaller than the distance D1 in FIG. 6a. For example, the projections 400 and 402 may be separated by a distance D2 in order to receive a relatively narrow blade 126 (FIG. 7b), and may be separated by a distance D3 in order to receive a wider blade 126 (FIG. 7c). Accordingly, the spacing of the projections 400 and 402 may be altered to receive a particular blade.

Referring to FIGS. 8a and 8b, in yet another embodiment, the arm 110 may be configured to receive a blade 126 (FIG. 8b) without the use of projections. The arm 110 may have the same channel 404 previously described (e.g., configured for a snap or tongue in groove fitting) or may be configured differently. Accordingly, the blade 126 may couple to the arm 110 in a manner similar to the projections.

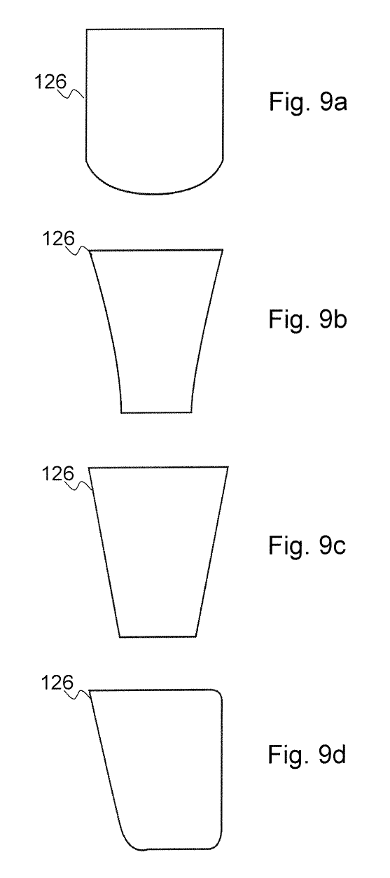

Referring to FIGS. 9a-9d, various embodiments of a blade 126 are illustrated. It is understood that many different variations are possible and the blade 126 is not limited to the illustrated examples. Furthermore, in other embodiments, multiple blades may be used with a single arm. For example, a separate blade may be attached to each projection of an arm.

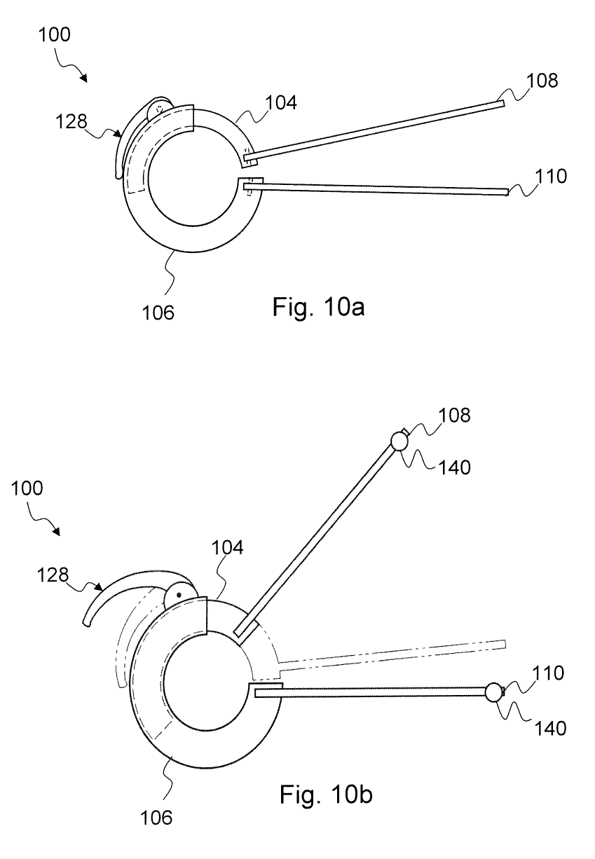

Referring to FIG. 10a, a top view of one embodiment of the retractor 100 of FIG. 1b is illustrated in a first or substantially closed position. In this position, a substantial part of the body portion 104 is exposed and the arms 108 and 110 are relatively close together. This position is reflective of substantially zero angle between the arms 108 and 110. Stated another way the arms 108 and 110 are parallel. It is understood that the body portion 104 may be extended until the exterior end 210 contacts the exterior end 222 of the body portion 106, which may indicate that the retractor 100 is fully closed. In this position the blades 124 and 126 are touching or are relatively close together and will therefore have substantially zero angle, or a very acute angle, between them. In the present example, the locking mechanism 128 is in the closed position, thereby preventing movement of the body portion 104 relative to the body portion 106.

Referring to FIG. 10b, a top view of one embodiment of the retractor 100 of FIG. 1b is illustrated in a second or substantially open position. In this position, a substantial portion of the body portion 104 is contained within the cavity of the body portion 106 and the arms 108 and 110 are relatively far apart or at a wide angle with respect to one another. It is understood that the body portion 104 may be retracted into the cavity until, for example, the arm 108 contacts the cavity end 220 of the body portion 106, which may indicate that the retractor 100 is fully open or encounters enough tissue resistance so as to halt opening. It can be seen that the body portions 104 and 106 will form only a portion of a circle, cylinder, or toroid (depending upon the particular embodiment) when the retractor 100 is open.

In the present example, the locking mechanism 128 is in the open position, thereby allowing movement of the body portion 104 relative to the body portion 106. The present embodiment also provides attachment points 140 which may be used for attaching an instrument (FIG. 17) to aid in opening the retractor 100.

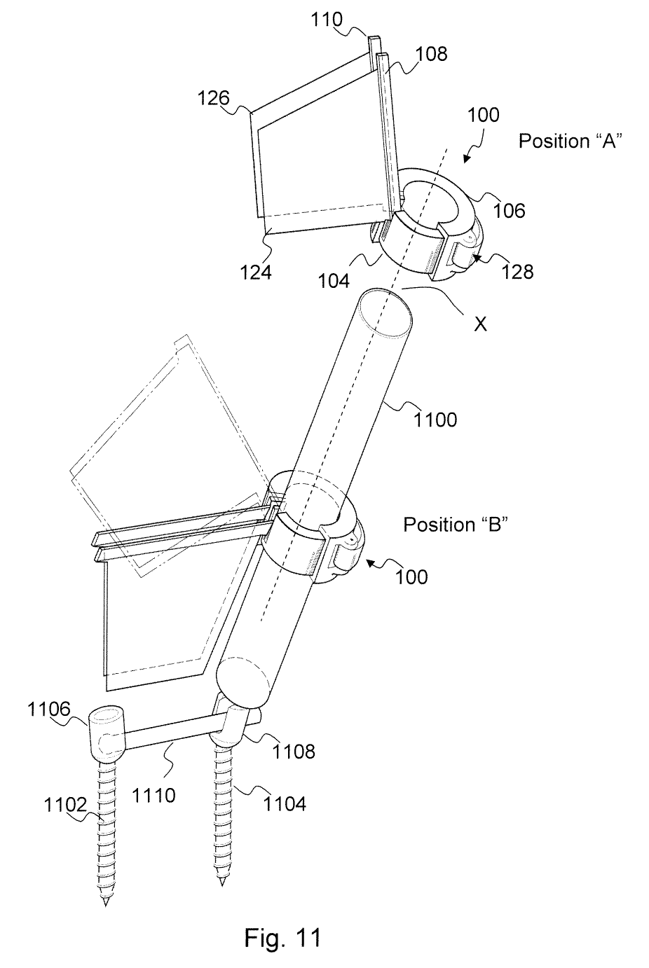

Referring to FIG. 11, in one embodiment, the retractor 100 of FIG. 1b is illustrated in conjunction with an extension 1100, such as may be used in a spinal surgery procedure. In the present example, bone anchors 1102 and 1104 (e.g., pedicle screws) may be fastened to a vertebral body (not shown). The bone anchors 1102 and 1104 may have polyaxial heads 1106 and 1108, respectively, which may be configured to receive a rod 1110. Examples of the extension 1100 and its interaction with the bone anchors 1102, 1104, polyaxial heads 1106 and 1108, and rod 1110 are illustrated in previously incorporated U.S. patent application Ser. Nos. 10/690,211; 10/990,272; and 10/989,715.

In operation, the retractor 100 may be placed in a position “A” above the extension 1100. As illustrated, the arms 108 and 110 may be rotated partially or totally upward, or angle toward the axis X. The circular body 102 of the retractor 100 may then be placed over the extension 1100 and may slide down the extension to a position “B”. At this point, the arms 108 and 110 may be lowered. In this position, the arms 108 and 110 may be substantially orthogonal to the axis X. It is noted that the locking mechanism 128 is down, indicating that the body portions 104 and 106 are locked with respect to one another. Accordingly, the opening 224 (FIG. 2) may be sized to slide over the extension 1100 regardless of the state of the retractor 100 (e.g., open or closed). However, in the present example, the opening 224 may only slide over the extension 1100 when the locking mechanism 128 is open.

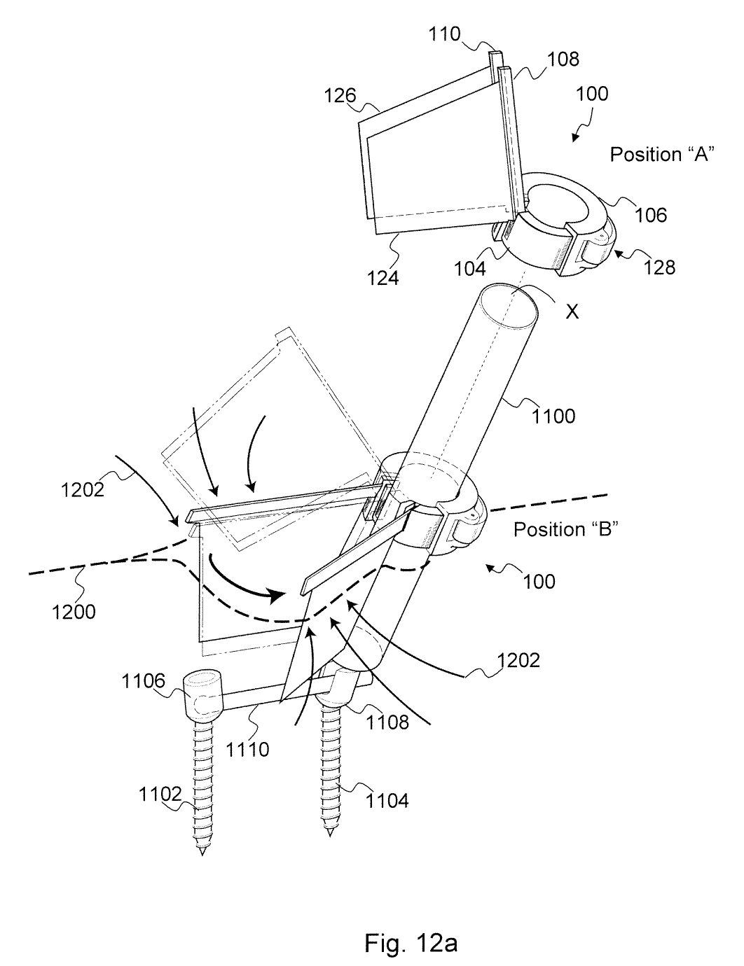

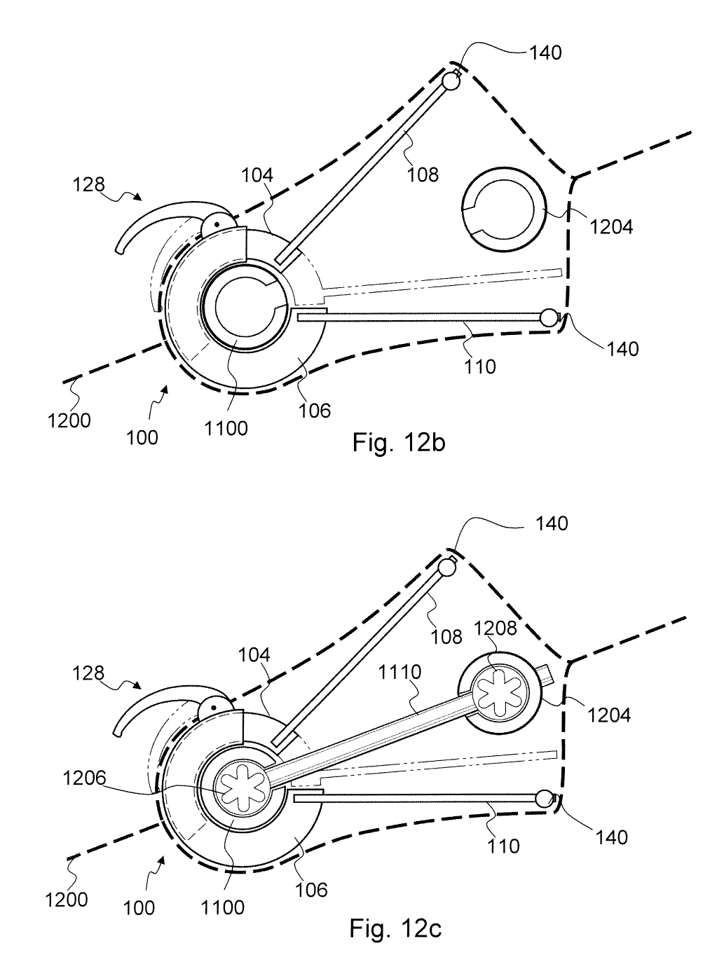

With additional reference to FIG. 12a, the retractor 100 of FIG. 1b is illustrated with a tissue layer 1200. For example, the tissue layer 1200 may be a layer of skin, with the extension 1100 positioned within an incision in the tissue layer. It is understood, however, that the blades 124 and 126 of the retractor 100 may be suitable for retraction of other tissue such as muscle and/or fat. As illustrated, the upper portion of the extension 1100 may be above the tissue layer 1200 and the lower portion below the tissue layer. Pressure, indicated by arrows 1202, may be applied by the tissue layer 1200 against the blades 124 and 126. The locking mechanism 128 may be used after opening the retractor 100 (e.g., moving the arm 108 relative to the arm 110) to counteract pressure 1202 from the tissue layer 1200 to keep the retractor open. Here is can be seen that the angle between the blades has been increased to allow access into the wound cavity.

Referring to FIG. 12b, a top view of one embodiment of the retractor 100 of FIG. 12a is illustrated. In the present example, a second extension 1204 is positioned over the bone anchor 1102 and polyaxial head 1106 (FIG. 12a). As illustrated, the arms 108 and 110 of the retractor 100 may be moved apart to provide an opening between the extensions 1100 and 1204.

Referring to FIG. 12c, the top view of FIG. 12b is illustrated with the rod 1110 in place between the polyaxial heads 1106 and 1108 (FIG. 12a). In the present example, to secure the rod 1110, a locking device 1206 may be inserted into the polyaxial head 1108 and a locking device 1208 may be inserted into the polyaxial head 1106. As such locking devices are described in the previously incorporated U.S. patent application Ser. Nos. 10/690,211; 10/990,272; and 10/989,715, they will not be described in detail herein.

With reference to FIGS. 12b-c, it can be seen how the angle between the blades 108 and 110 corresponds to varying degrees of retraction. The relationship of the blades 108 and 110 when in the original, closed position can be seen in dotted line. In order to assist in opening the retractor, attachment points 140 are provided. In some embodiments, an instrument (FIG. 17) may be used to apply outward force to the blades to provide the appropriate angle therebetween for effective retraction. As described, the retractor 100 can then be locked into the open position. The attachment points 140 may be knobs or stems on the arms 108 and 110 or blades 124 and 126.

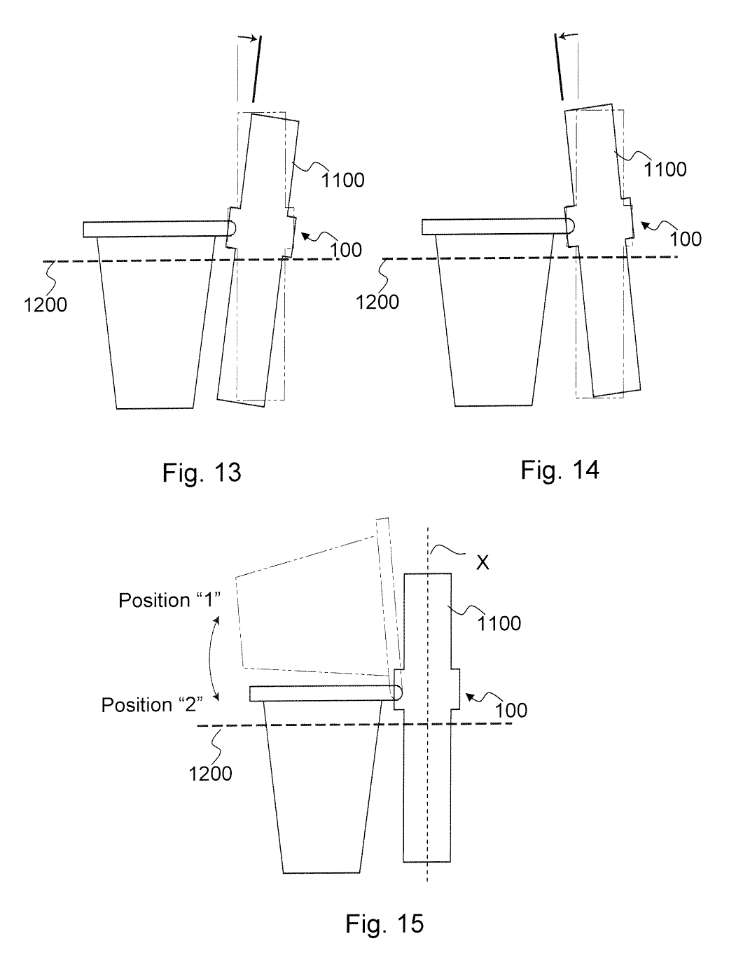

Referring to FIGS. 13-15, various embodiments of the retractor 100 of FIG. 11 illustrate possible orientations of the retractor based on the orientation of the extension 1100. For example, in FIG. 13, the extension 1100 may have an angled orientation (indicated by solid lines) wherein the arms 108 and 110 form an obtuse angle with the upper portion of the extension as compared to a vertical orientation (indicated by dashed lines). In FIG. 14, the extension 1100 may have an angled orientation (indicated by solid lines) wherein the arms 108 and 110 form an acute angle with the upper portion of the extension as compared to a vertical orientation (indicated by dashed lines). FIG. 15 illustrates a vertical orientation wherein the arms 108 and 110 form a substantially ninety degree angle with the upper portion of the extension when the arms are lowered from a first position “1” to a second position “2”. It is noted that the arms 108 and 110 may remain substantially parallel to the tissue layer 1200 throughout the different orientations illustrated in FIGS. 13-15. In addition, the arms 108 and 110 may remain together or they may be placed separately.

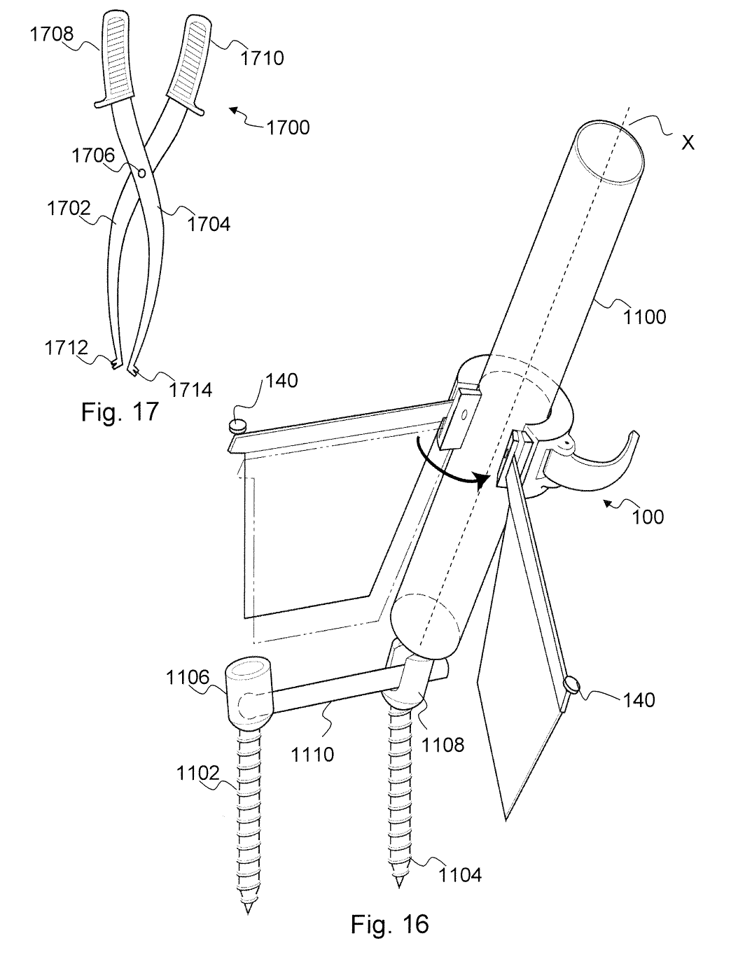

Referring to FIG. 16, the retractor 100 of FIG. 11 is illustrated in position “B” with the locking mechanism 128 having been opened to allow movement of the body portion 104 relative to the body portion 106. In this opened configuration, the arms and blades may be adjusted between closed, acute, and obtuse angles.

With additional reference to FIG. 17, a device 1700 illustrates one embodiment of an instrument that may be used to force apart the arms 108 and 110 and their respective blades 124 and 126. In the present example, the device 1700 includes opposing arms 1702 and 1704 that are joined by a pin 1706. User gripping surfaces 1708 and 1710 may be provided for the opposing arms 1702 and 1704, respectively. Ends 1712 and 1714 of the opposing arms 1702 and 1704, respectively, may be configured to engage the attachment points 140, the body portions 104 and 106, the arms 108 and 110, and/or blades 124 and 126.

Referring again to FIG. 16, using an instrument such as the device 1700 of FIG. 17, the arms 108 and 110 of the retractor 100 may be moved from a substantially closed position (FIGS. 11 and 12) to the illustrated substantially open position. When open, the blades 124 and 126 may create a void within the tissue to allow access to a surgical site. It is understood that the arms 108 and 110 may be stopped at any position between fully closed and fully open, and the positions illustrated herein are not limiting. Once the arms 108 and 110 are in the desired position, the locking mechanism 128 may be closed (not shown) to prevent further movement of the body portions 104 and 106. This may prevent the arms 108 and 110 from being forced together by the pressure asserted by the surrounding tissue and may lock the retractor 100 onto the extension 1100.

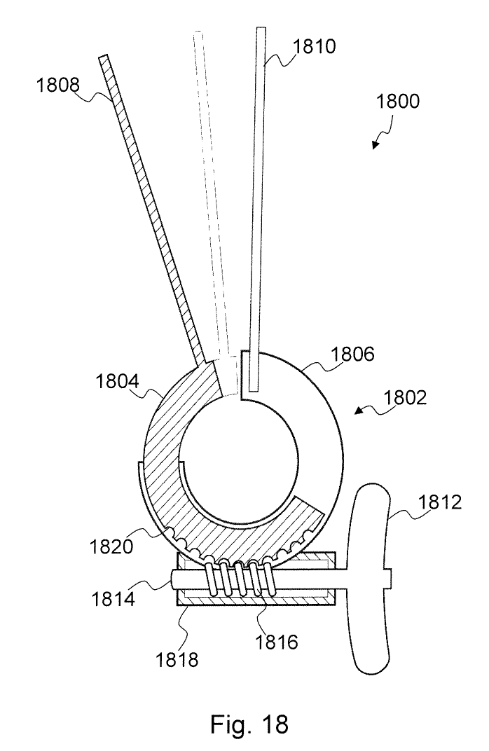

Referring to FIG. 18, in another embodiment, a retractor 1800 may include a different mechanism for opening and closing than that discussed above. In the present example, the retractor 1800 may include a substantially circular body 1802 formed by semi-circular body portions 1804 and 1806. The body portions 1804 and 1806 may move relative to one another. For example, the body portion 1806 may include a cavity large enough to receive at least part of the body portion 1804. Accordingly, the body portions 1804 and 1806 may move with respect to one another, and such movement may be restrained to a substantially circular path. In the present embodiment, the body portion 1804 may not be removable from the body portion 1806 due to a length of the body portion 1804 captured within the cavity of the body portion 1806. The body portions 1804 and 1806 may be coupled to arms 1808 and 1810, respectively, which extend outwardly from the circular body 1802.

In the present embodiment, a driver 1812 may be coupled to a shaft 1814. The shaft 1814 may include threads 1816 and may be contained within a sleeve or housing 1818. The outer surface of the body portion 1804 may include a series of depressions 1820 that engage the threads 1816. The body portion 1806 may or may not contain a means for engaging the threads 1816.

In operation, as the driver 1812 is rotated, the shaft 1814 and the associated threads 1816 are also rotated. The threads 1816 engage the depressions 1820 in the body portion 1804 and cause the body portion to rotate. The direction of rotation may depend on the direction of rotation of the driver. Accordingly, using the mechanism illustrated, the arms 1808 and 1810 may be moved together or apart using the driver 1812. It can be seen that the shaft 1814 and threads 1816 may operate as a worm gear to move the body portion 1814 relative to the body portion 1806.

In such an embodiment as shown in FIG. 18, an instrument such as the device 1700 of FIG. 17 may not be needed. Furthermore, in embodiments where there is sufficient friction within the retractor 1800, a locking means such as the locking mechanism 128 of FIG. 1b may not be needed.

It is understood that a variety of materials may be used to form the various components of the retractors described herein. For example, various metals, alloys, plastics, and other materials may be used to create a retractor, and various portions of the retractor may be formed from different materials. In another example, a material such as cloth or mesh may be coupled to the distal ends 114 and 118 of the arms 108 and 110, respectively, and the material may form a third side when the arms are separated. In operation, the material may be folded between the blades 124 and 126 when the blades are initially inserted into the surgical site, and the material may then be pulled tight to couple the distal ends 114 and 118 as the blades are separated.

The foregoing description of the embodiments of the invention has been presented for the purposes of illustration and description. It is not intended to be exhaustive or to limit the invention to the precise form disclosed. Many modifications and variations are possible in light of the above teaching. For example, various elements of the embodiments described above may be combined with elements of other embodiments, and characteristics of one embodiment may be incorporated into another embodiment. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps, and it is intended that the scope of the invention not be limited by this detailed description.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A kit for performing back surgery, the kit comprising:
a first bone anchor;
a second bone anchor;
an extension having a proximal end portion and a distal end portion, wherein the distal portion is adapted to couple to at least one of the bone anchors;
a plurality of blades; and
a retractor, including:
a body, with first and second semi-circular portions that are circularly displaceable with respect to one another about an axis extending through the body and forming an aperture sized to allow a portion of the extension to be received within the aperture;
first and second arms attached to the first and second body portions, respectively, by hinged couplings allowing rotation about axes that are substantially orthogonal to the axis extending through the body, wherein the first and second arms are each adapted to couple to at least one blade of the plurality of blades, wherein the circular displacement of the first and second body portions defines an angle between the first and second blades; and
a locking mechanism having a first position and a second position, wherein in a first position the angle between the first and second blades is locked, and in a second position, the angle between the first and second blades is not locked.

2. The kit of claim 1 wherein the plurality of blades include blades of differing shapes and sizes.

3. The kit of claim 1 wherein a portion of the extension couples to a portion of the retractor body in a tongue-in-groove manner.

4. The kit of claim 1 wherein each of the first and second arms comprise at least one projection projecting transverse to the arm and wherein the each of the plurality of the blades has at least one slot for receiving the corresponding at least one projection.

5. The kit of claim 1 further comprising a tool having a distal portion and a proximal portion, wherein the distal portion is adapted to couple to the blades such that the tool can apply pressure to the blades to adjust the angle therebetween.

6. A surgical retractor comprising:
a body with first and second portions cooperating to displace circularly relative to one another about an axis through a center of the body;
a first arm coupled to the first body portion and a second arm coupled to the second body portion;
a first retractor blade coupled to the first arm; and
a second retractor blade coupled to the second arm;
wherein the first and second retractor blades extend radially from the axis of the retractor body and define an angle therebetween, wherein the angle is adjusted by the circular displacement of the first and second body portions.

7. The surgical retractor of claim 6, further comprising:
a locking mechanism selectively locking the first and second body portions together to prevent circular displacement therebetween, the locking mechanism coupled to the first and second body portions.

8. The surgical retractor of claim 6,
wherein the retractor blades rotate about axes that are substantially orthogonal to the axis through the center of the body.

9. The surgical retractor of claim 6, wherein the first and second blades are removable from the first and second arms to allow replacement and exchange of the first and second blades.

10. A surgical retractor comprising:
a retractor body having a first portion, a second portion, and a central axis;
a first arm coupled to the first portion of the retractor body;
a second arm coupled to the second portion of the retractor body;
a first blade coupled to the first arm and extending radially outward from the central axis of the retractor body; and
a second blade coupled to the second arm and extending radially outward from the central axis of the retractor body, wherein the first and second blades define an angle therebetween;
wherein the first portion of the retractor body is slidably receivable by the second portion of the body and wherein sliding of the first portion with respect to the second portion adjusts the angle between the first and second blades; and
wherein the first and second arms are rotationally displaceable around axes substantially perpendicular to the central axis.

11. The surgical retractor of claim 10, further comprising:
a locking mechanism adapted to lock the first and second body portions and maintain the angle between the blades; the locking mechanism coupled to the first and second body portions.

12. The surgical retractor of claim 11, wherein the locking mechanism is a cam lock.

13. The surgical retractor of claim 10, further comprising:
a worm gear coupled to the second body portion and engaging a series of indentations on the first portion of the retractor body.

14. The surgical retractor of claim 10, further comprising:
a first plurality of projections extending from the first arm coupling the first blade to the first arm; and
a second plurality of projections extending from the second arm coupling the second blade to the second arm.

15. The surgical retractor of claim 14, wherein each of the first and second plurality of projections is movable along a length of each respective arm for receiving blades of varying size.

16. A surgical retractor comprising:
a body with first and second portions that are circularly displaceable with respect to one another about an axis extending through the body; and
a first arm coupled to the first body portion and extending radially outward from the axis extending through the body;
a second arm coupled to the second body portion and extending radially outward from the axis extending through the body, wherein the circular displacement of the first and second body portions defines an angle between the first and second arms; and
a first and second blade coupled to the first and second arms, respectively, wherein the first and second blades are each rotatable about an axis that is substantially orthogonal to the axis extending through the body.

17. The surgical retractor of claim 14, further comprising:
a locking mechanism adapted to maintain the relative positions between the first and second body portions; the locking mechanism coupled to the first and second body portions.

18. The surgical retractor of claim 15, wherein the locking mechanism comprises:
a levered cam on the second body portion capable of applying restrictive pressure on the first body portion.

19. The surgical retractor of claim 15, wherein the locking mechanism comprises:
a worm gear coupled to the second body portion interfacing with a series of teeth on the first body portion.

20. The surgical retractor of claim 14, wherein the first and second blades are interexchangeable with a plurality of blades of differing shape.

21. A method for retracting tissues in a surgical procedure, comprising:
providing a retractor device having a body with a central axis, a first body portion coupled to a second body portion, a first arm having a first blade and extending radially outward from the first body portion, and a second arm having a second blade and extending radially outward from the second body portion, wherein an angle is defined between the first and second blades;
implanting a bone anchor into a bone structure of a patient;
securing an extension to the implanted anchor;
sliding the retractor over the implanted anchor extension;
rotating the first arm of the retractor relative to the second arm of the retractor in a plane substantially perpendicular to a longitudinal axis of the extension, such that the first body portion of the retractor rotates relative to the second body portion of the retractor; and
locking the first body portion relative to the second body portion, such that the first arm is locked relative to the second arm.

22. The method of claim 21 further comprising:
pivoting the first arm of the retractor around an axis that is substantially perpendicular to the central axis of the retractor; and
pivoting the second arm of the retractor around an axis that is substantially perpendicular to the central axis of the retractor.

23. The method of claim 21 wherein rotating the first arm of the retractor relative to the second arm of the retractor further comprises:
angularly adjusting the first blade of the retractor relative to the second blade the retractor.

24. The method of claim 23 wherein rotating the first arm of the retractor relative to the second arm of the retractor further comprises:
coupling a blade moving mechanism to the first blade and the second blade;
angularly adjusting the first blade relative to the second blade by activating the blade moving mechanism; and
uncoupling the blade moving mechanism from the first and second blades.

25. The method of claim 21 wherein sliding the retractor over the extension further comprises:
coupling the retractor to the extension in a tongue and groove manner.
